(19) United States
(12) Patent Application Publication
(10) Pub. No.: US 2006/0147458 A1
(43) Pub. Date: Jul. 6, 2006

(54) Vaccines

(76) Inventors: Paul Andrew Hamblin, Stevenage (GB); Maria Des los Angeles Rocha Del Cura, Stevenage (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/547,207

(22) PCT Filed: Feb. 26, 2004
(86) PCT No.: PCT/EP04/02007

§ 371 Date: Aug. 26, 2005

(30) Foreign Application Priority Data
Feb. 28, 2003 (GB) 0304634.9

(51) Int. Cl. A61K 39/00; C07H 21/04; C12P 21/06; C07K 14/82

(52) U.S. Cl. 424/185.1; 435/69.1; 435/320.1; 435/325; 530/350; 536/23.5; 424/489

(57) ABSTRACT

Novel MUC-1 DNA constructs are provided that have reduced homology to native MUC-1. Pharmaceutical compositions containing such MUC-1 constructs are provided.

The present invention relates to the novel nucleic acid constructs, useful in nucleic acid vaccination protocols for the treatment and prophylaxis of MUC-1 expressing tumours. In particular, the nucleic acid is DNA and the DNA constructs comprise a gene encoding a MUC-1 derivative optionally devoid of all the perfect repeats. More particularly, the nucleic acid is modified to minimise the homology to wild type Muc-1. The invention further provides pharmaceutical compositions comprising said constructs, particularly pharmaceutical compositions adapted for particle mediated delivery, methods for producing them, and their use in medicine.

### BACKGROUND TO THE INVENTION

The epithelial cell mucin MUC-1 (also known as episialin or polymorphic epithelial mucin, PEM) is a large molecular-weight glycoprotein expressed on many epithelial cells. The protein consists of a cytoplasmic tail, a transmembrane domain and a variable number of tandem repeats of a 20 amino acid motif (herein termed the VNTR monomer, it may also be known as the VNTR epitope, or the VNTR repeat) containing a high proportion of proline, serine and threonine residues. The number of repeats is variable due to genetic polymorphism at the MUC-1 locus, and most frequently lies within the range 30-100 (Swallow et al, 1987, Nature 328:82-84). In normal ductal epithelia, the MUC-1 protein is found only on the apical surface of the cell, exposed to the duct lumen (Graham et al, 1996, Cancer Immunol Immunother 42:71-80; Barratt-Boyes et al, 1996, Cancer Immunol Immunother 43:142-151). One of the most striking features of the MUC-1 molecule is its extensive O-linked glycosylation. There are five O-linked glycosylation sites available within each MUC-1 VNTR monomer.

The VNTR can be characterised as typical or perfect repeats and imperfect (atypical) repeats which has minor variation for the perfect repeat comprising two to three differences over the 20 amino acids. The following is the sequence of the perfect repeat.

```
1 2 3 4 5 6 7 8 9 10 11 12 13 14 15 16 17 18 19 20
A P D T R P A P G S T A P P A H G V T S
        E S T
                                                            A
                                                            Q
```

Amino acids that are underlined may be substituted for the amino acid residues shown. The perfect repeat is an identical repeated sequence with the exception of the defined amino acid substitutions (ie D to E at position 3, T to S at position 4 and P to T, A or Q at position 14. Perfect repeats may be characterised by the fact that they can be represented many times within a single MUC1 molecule.

Imperfect repeats have different amino acid substitutions to the consensus sequence above with 55-90% identity at the amino acid level. The four imperfect repeats are shown below, with the substitutions underlined:

| | |
|---|---|
| APDTRPAPGSTAPPAHGVTS | perfect repeat |
| APATEPASGSAATWGQDVTS | imperfect repeat 1 |
| VPVTRPALGSTTPPAHDVTS | imperfect repeat 2 |
| APDNKPAPGSTAPPAHGVTS | imperfect repeat 3 |
| APDNRPALGSTAPPVHNVTS | imperfect repeat 4 |

The imperfect repeat in wild type—Muc-1 flank the perfect repeat region. Each different imperfect repeat is generally represented only once in the MUC1 sequence and shows between 2 and 9 amino acid substitutions from the perfect repeat sequence (which equates to between 55-90% amino acid identity).

In malignant carcinomas arising by neoplastic transformation of these epithelial cells, several changes affect the expression of MUC-1. The polarised expression of the protein is lost, and it is found spread over the whole surface of the transformed cell. The total amount of MUC-1 is also increased, often by 10-fold or more (Strous & Dekker, 1992, Crit Rev Biochem Mol Biol 27:57-92). Most significantly, the quantity and quality of the O-linked carbohydrate chains changes markedly. Fewer serine and threonine residues are glycosylated. Those carbohydrate chains that are found are abnormally shortened, creating the tumour-associated carbohydrate antigen STn (Lloyd et al, 1996, J Biol Chem, 271:33325-33334). As a result of these glycosylation changes, various epitopes on the peptide chain of MUC-1 which were previously screened by the carbohydrate chains become accessible. One epitope which becomes accessible in this way is formed by the sequence APDTR (Ala 8-Arg 12) present in each 20 amino acid VNTR perfect monomer (Burchell et al, 1989, Int J Cancer 44:691-696).

It is apparent that these changes in MUC-1 mean that a vaccine that can activate the immune system against the form of MUC-1 expressed on tumours may be effective against epithelial cell tumours, and indeed other cell types where MUC-1 is found, such as T cell lymphocytes. One of the main effector mechanisms used by the immune system to kill cells expressing abnormal proteins is a cytotoxic T lymphocyte immune response (CTL's) and this response is desirable in a vaccine to treat tumours, as well as an antibody response. A good vaccine will activate all arms of the immune response. However, current carbohydrate and peptide vaccines such as Theratope or BLP25 (Biomira Inc, Edmonton, Canada) preferentially activate one arm of the immune response—a humoral and cellular response respectively, and better vaccine designs are desirable to generate a more balanced response.

Nucleic acid vaccines provide a number of advantages over conventional protein vaccination, in that they are easy to produce in large quantity. Even at small doses they have been reported to induce strong immune responses, and can induce a cytotoxic T lymphocyte immune response as well as an antibody response.

The full-length MUC-1, however, is very difficult to work with due to the highly repetitive sequence, since it is highly susceptible to recombination, such recombination events cause significant development difficulties. Additionally the GC rich nature of the VNTR region makes sequencing difficult. Further for regulatory reasons—it is necessary to fully characterise the DNA construct. It is highly problematic to sequence a molecule with such a high frequency repeating structure. Given that it is unknown precisely how many repeat units are in wild type MUC-1 this inability to precisely characterise full-length MUC-1 makes this unacceptable for regulatory approval.

### SUMMARY OF THE INVENTION

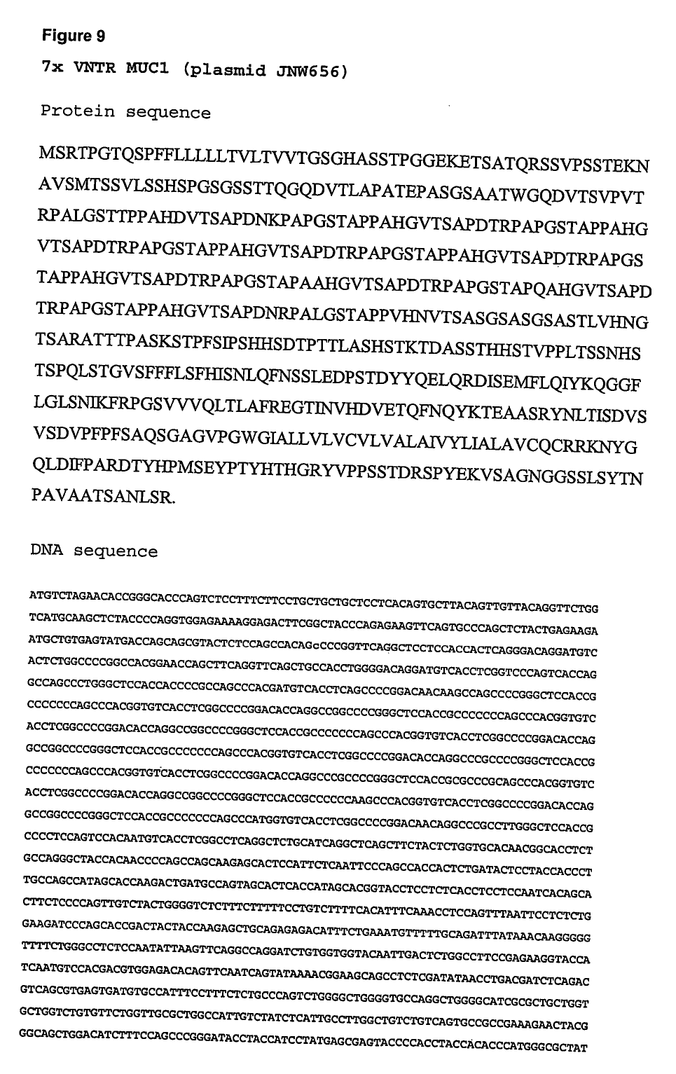

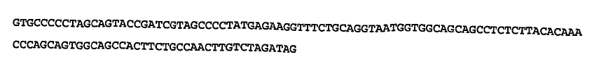

The present invention provides a nucleic acid sequence encoding a MUC-1 derivative which is capable of raising an immune response in vivo, said immune response being capable of recognising a MUC-1 expressing tumour, wherein the nucleic acid is modified such that the non-repeat region has a RSCU of at least 0.6, and has a level of identity with respect to wild type MUC-1 DNA over the corresponding non-repeat regions of less than 85% in comparison with the MUC-1 VNTR nucleotide sequence shown in FIG. 9.

In one embodiment, the nucleic acid encodes for a MUC-1 derivative as described above devoid of any repeat (both perfect and imperfect) units.

In an alternative embodiment, the nucleic acid sequence is devoid of only the perfect repeats. In yet a further embodiment, the nucleic acid construct contains between 1 and 15 perfect repeats, preferably 7 perfect repeats. The perfect repeat may or may not be modified from the wild type MUC-1.

The non-perfect repeat region in a more preferred embodiment has a RSCU (Relative synomons Codon useage (also known as Codon Index CI)) of at least 0.65 and less than 80% identity to the non-perfect repeat region.

Such constructs, are surprisingly, capable of raising both a cellular and also an antibody response that recognise MUC-1 expressing tumour cells.

The constructs can also contain altered repeat (VNTR units) such as reduced glycosylation mutants. Foreign T-cell epitopes that may be incorporated include T-helper epitopes such as derived from bacterial proteins and toxins and from viral sources, eg. T-Helper epitopes from Diphtheria or Tetanus, eg P2 and P30 or epitopes from Hep B core antigen. These maybe incorporated within or at either end of the MUC-1 constructs of the invention.

In yet further embodiments, the invention contemplates nucleic acids that encode for fusion proteins that have heterologous protein at the N or C terminus of the MUC-1 constructs of the invention. Such fusion partners, provide T-helper epitopes or are capable of eliciting a re-call response.

Examples of these include Tetanus, Diptheria, Tuberculosis or hepatitis proteins, such as Tetanus or Diptheria toxin, in particular a fragment of Tetanus toxin that incorporates the P2 and/or P30 epitope. An example of a Mycobacterium tuberculosis peptide is Ra12 corresponding to amino-acids 192 to 323 of Mtb32a (Skeiky et al Infection and Immunity (1999) 67: 3998-4007). Hepatitis B core antigen is illustrative of yet another embodiment.

Other preferred immunological fusion partners include protein D, typically the N terminal 1/3 (eg N terminal 1-109); LYTA or portion thereof (preferably the C-terminal portion) from Streptococcus pneumoniae (Biotechnology) 10: 795-798, 1992).

In further aspect of the invention the nucleic acid sequence is a DNA sequence in the form of a plasmid. Preferably the plasmid is super-coiled. Proteins encoded by such nucleotide sequences are novel and form an aspect of the invention.

In a further aspect of the invention there is provided a pharmaceutical composition comprising a nucleic acid sequence or protein as herein described and a pharmaceutical acceptable excipient, diluent or carrier.

Preferably the carrier is a gold bead and the pharmaceutical composition is amenable to delivery by particle mediated drug delivery.

In yet a further embodiment, the invention provides the pharmaceutical composition and nucleic acid constructs for use in medicine. In particular, there is provided a nucleic acid construct of the invention, in the manufacture of a medicament for use in the treatment or prophylaxis of MUC-1 expressing tumours.

The invention further provides for methods of treating a patient suffering from or susceptible to MUC-1 expressing tumour, particularly carcinoma of the breast, lung, prostate (particularly non-small cell lung carcinoma), gastric and other GI (gastrointestinal) carcinomas by the administration of a safe and effective amount of a composition or nucleic acid as herein described.

In yet a further embodiment the invention provides a method of producing a pharmaceutical composition as herein described by admixing a nucleic acid construct or protein of the invention with a pharmaceutically acceptable excipient, diluent or carrier.

### DETAILED DESCRIPTION OF THE INVENTION

The wild type MUC-1 molecule contains a signal sequence, a leader sequence, imperfect or atypical VNTR, the perfect VNTR region, a further atypical VNTR, a non-VNTR extracellular domain a transmembrane domain and a cytoplasmic domain.

Constructs are provided wherein the non-VNTR region are codon modified to have a RSCU of at least 0.6 and having less than 85% identity to the corresponding wild type region. Such constructs are advantageous—as they reduce the potential of homologous recombination, have enhanced expression and are immunogenic and capable of raising both a cellular and antibody response that recognise MUC-1 expressing tumour cells.

More preferably the regions codon modified have a RSCU of at least 0.65 and have less that 80% identity to the corresponding wild type region. When comparing polynucleotide sequences, two sequences are said to be “identical” if the sequence of nucleotides in the two sequences is the same when aligned for maximum correspondence, as described below.

Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Thus in the present invention, the non-repeat region of the codon-modified and the non-repeat region of optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information.

The DNA code has 4 letters (A, T, C and G) and uses these to spell three letter “codons” which represent the amino acids the proteins encodes in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In consequence, codons preferred by a particular prokaryotic (for example E. coli or yeast) or eukaryotic host can be modified so as to encode the same protein, but to differ from a wild type sequence. The process of codon modification may include any sequence, generated either manually or by computer software, where some or all of the codons of the native sequence are modified. Several method have been published (Nakamura et. al., Nucleic Acids Research 1996, 24:214-215; WO98/34640). One preferred method according to this invention is Syngene method, a modification of Calcgene method (R. S. Hale and G Thompson (Protein Expression and Purification Vol. 12 pp. 185-188 (1998)).

This process of codon modification may have some or all of the following benefits: 1) to improve expression of the gene product by replacing rare or infrequently used codons with more frequently used codons, 2) to remove or include restriction enzyme sites to facilitate downstream cloning and 3) to reduce the potential for homologous recombination between the insert sequence in the DNA vector and genomic sequences and 4) to improve the immune response in humans. The sequences of the present invention advantageously have reduced recombination potential, but express to at least the same level as the wild type sequences. Due to the nature of the algorithms used by the SynGene programme to generate a codon modified sequence, it is possible to generate an extremely large number of different codon modified sequences which will perform a similar function. In brief, the codons are assigned using a statistical method to give synthetic gene having a codon frequency closer to that found naturally in highly expressed human genes such as β-Actin.

In the polynucleotides of the present invention, the codon usage pattern is altered from that typical of MUC-1 to more closely represent the codon bias of a highly expressed gene in a target organism, for example human β-actin. The “codon usage coefficient” is a measure of how closely the codon pattern of a given polynucleotide sequence resembles that of a target species. Codon frequencies can be derived from literature sources for the highly expressed genes of many species (see e.g. Nakamura et. al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species. According to the present invention, the codon usage pattern of the polynucleotide will preferably exclude codons representing <10% of the codons used for a particular amino acid. A relative synonymous codon usage (RSCU) value is the observed number of codons divided by the number expected if all codons for that amino acid were used equally frequently. A polynucleotide of the present invention will preferably exclude codons with an RSCU value of less than 0.2 in highly expressed genes of the target organism. A polynucleotide of the present invention will generally have a codon usage coefficient for highly expressed human genes of greater than 0.6, preferably greater than 0.65, most preferably greater than 0.7. Codon usage tables for human can also be found in Genbank.

In comparison, a highly expressed beta actin gene has a RSCU of 0.747.

The codon usage table for a homo sapiens is set out below:

Codon usage for human (highly expressed)
genes Jan. 24, 1991 (human_high.cod)

| AmAcid | Codon | Number | /1000 | Fraction | . . . |
|---|---|---|---|---|---|
| Gly | GGG | 905.00 | 18.76 | 0.24 | |
| Gly | GGA | 525.00 | 10.88 | 0.14 | |
| Gly | GGT | 441.00 | 9.14 | 0.12 | |
| Gly | GGC | 1867.00 | 38.70 | 0.50 | |
| Glu | GAG | 2420.00 | 50.16 | 0.75 | |
| Glu | GAA | 792.00 | 16.42 | 0.25 | |
| Asp | GAT | 592.00 | 12.27 | 0.25 | |
| Asp | GAC | 1821.00 | 37.75 | 0.75 | |
| Val | GTG | 1866.00 | 38.68 | 0.64 | |
| Val | GTA | 134.00 | 2.78 | 0.05 | |
| Val | GTT | 198.00 | 4.10 | 0.07 | |
| Val | GTC | 728.00 | 15.09 | 0.25 | |
| Ala | GCG | 652.00 | 13.51 | 0.17 | |
| Ala | GCA | 488.00 | 10.12 | 0.13 | |
| Ala | GCT | 654.00 | 13.56 | 0.17 | |
| Ala | GCC | 2057.00 | 42.64 | 0.53 | |
| Arg | AGG | 512.00 | 10.61 | 0.18 | |
| Arg | AGA | 298.00 | 6.18 | 0.10 | |
| Ser | AGT | 354.00 | 7.34 | 0.10 | |
| Ser | AGC | 1171.00 | 24.27 | 0.34 | |
| Lys | AAG | 2117.00 | 43.88 | 0.82 | |
| Lys | AAA | 471.00 | 9.76 | 0.18 | |
| Asn | AAT | 314.00 | 6.51 | 0.22 | |
| Asn | AAC | 1120.00 | 23.22 | 0.78 | |
| Met | ATG | 1077.00 | 22.32 | 1.00 | |
| Ile | ATA | 88.00 | 1.82 | 0.05 | |
| Ile | ATT | 315.00 | 6.53 | 0.18 | |
| Ile | ATC | 1369.00 | 28.38 | 0.77 | |
| Thr | ACG | 405.00 | 8.40 | 0.15 | |
| Thr | ACA | 373.00 | 7.73 | 0.14 | |
| Thr | ACT | 358.00 | 7.42 | 0.14 | |
| Thr | ACC | 1502.00 | 31.13 | 0.57 | |
| Trp | TGG | 652.00 | 13.51 | 1.00 | |
| End | TGA | 109.00 | 2.26 | 0.55 | |
| Cys | TGT | 325.00 | 6.74 | 0.32 | |
| Cys | TGC | 706.00 | 14.63 | 0.68 | |
| End | TAG | 42.00 | 0.87 | 0.21 | |
| End | TAA | 46.00 | 0.95 | 0.23 | |
| Tyr | TAT | 360.00 | 7.46 | 0.26 | |
| Tyr | TAC | 1042.00 | 21.60 | 0.74 | |
| Leu | TTG | 313.00 | 6.49 | 0.06 | |
| Leu | TTA | 76.00 | 1.58 | 0.02 | |
| Phe | TTT | 336.00 | 6.96 | 0.20 | |
| Phe | TTC | 1377.00 | 28.54 | 0.80 | |
| Ser | TCG | 325.00 | 6.74 | 0.09 | |
| Ser | TCA | 165.00 | 3.42 | 0.05 | |
| Ser | TCT | 450.00 | 9.33 | 0.13 | |
| Ser | TCC | 958.00 | 19.86 | 0.28 | |
| Arg | CGG | 611.00 | 12.67 | 0.21 | |
| Arg | CGA | 183.00 | 3.79 | 0.06 | |
| Arg | CGT | 210.00 | 4.35 | 0.07 | |
| Arg | CGC | 1086.00 | 22.51 | 0.37 | |
| Gln | CAG | 2020.00 | 41.87 | 0.88 | |
| Gln | CAA | 283.00 | 5.87 | 0.12 | |
| His | CAT | 234.00 | 4.85 | 0.21 | |
| His | CAC | 870.00 | 18.03 | 0.79 | |
| Leu | CTG | 2884.00 | 59.78 | 0.58 | |
| Leu | CTA | 166.00 | 3.44 | 0.03 | |
| Leu | CTT | 238.00 | 4.93 | 0.05 | |
| Leu | CTC | 1276.00 | 26.45 | 0.26 | |
| Pro | CCG | 482.00 | 9.99 | 0.17 | |
| Pro | CCA | 456.00 | 9.45 | 0.16 | |
| Pro | CCT | 568.00 | 11.77 | 0.19 | |
| Pro | CCC | 1410.00 | 29.23 | 0.48 | |

The non-VNTR extracellular domain is approximately 80 amino acids, 5′ of VNTR and 190-200 amino acids 3′ VNTR. All constructs of the invention comprise at least one epitope from this region. An epitope is typically formed from at least seven amino acid sequence. Accordingly the constructs of the present invention include at least one epitope from the non VNTR extra-cellular domain. Preferably substantially all or more preferably all of the non-VNTR domain is included. It is particularly preferred that construct contains the epitope comprised by the sequence FLSFHISNL; NSSLEDPSTDYYQELQRDISE, or NLTISDVSV. More preferred is that two, preferable all three, epitope sequences are incorporated in the construct.

In a preferred embodiment the constructs comprise an N-terminal leader sequence. The signal sequence, transmembrane domain and cytoplasmic domain are individually all optionally present or deleted. When present it is preferred that all these regions are modified.

Preferred constructs according to the invention are:

1) Codon modified truncated MUC-1 (ie Full MUC-1 with no perfect repeats)

2) Codon modified truncated MUC-1 Δss (As I, but also devoid of signal sequence)

3) Codon modified truncated MUC-1 ΔTM ΔCYT (As 1, but devoid of Transmembrane and cytoplasmic domains)

4) Codon modified truncated MUC-1 Δss ΔTM ΔCYT (As 3, but also devoid of signal sequence)

Also preferred are equivalent constructs of 1 to 4 above, but devoid of imperfect MUC-1 repeat units. Such constructs are referred to as gutted-MUC-1. In an embodiment one or more of the imperfect VNTR units is mutated to reduce the potential for glycosylation, by altering a glycosylation site. The mutation is preferably a replacement, but can be an insertion or a deletion. Typically at least one threonine or serine is substituted with valine, Isoleucine, alanine or asparagine. It is thus preferred that at least one, preferably 2 or 3 or more are substituted with an amino acid as noted above.

Other preferred constructs are the equivalent to the above, but comprising from 1-15, preferably 2-8, most preferably 7 VNTR (perfect) repeat units.

In a further embodiment, the gutted MUC-1 nucleic acid is provided with a restriction site at the junction of the leader sequence and the extracellular domain. Typically this restriction site is a NheI site. This can be utilised as a cloning site to insert sequences encoding for other peptides including, for example glycosylation mutants (ie. VNTR regions mutated to remove O-glycosylation sites), or heterologous sequences that encode T-Helper epitopes such as P2 or P30 from Tetanus toxin, or wild type VNTR units.

According to a further aspect of the invention, an expression vector is provided which comprises and is capable of directing the expression of a polynucleotide sequence according to the invention. The vector may be suitable for driving expression of heterologous DNA in bacterial insect or mammalian cells, particularly human cells.

According to a further aspect of the invention, a host cell comprising a polynucleotide sequence according to the invention, or an expression vector according the invention is provided. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

The present invention further provides a pharmaceutical composition comprising a polynucleotide sequence according to the invention. Preferably the composition comprises a DNA vector. In preferred embodiments the composition comprises a plurality of particles, preferably gold particles, coated with DNA comprising a vector encoding a polynucleotide sequence of the invention which the sequence encodes a MUC-1 amino acid sequence as herein described. In alternative embodiments, the composition comprises a pharmaceutically acceptable excipient and a DNA vector according to the present invention.

The composition may also include an adjuvant, or be administered either concomitantly with or sequentially with an adjuvant or immuno-stimulatory agent.

Thus it is an embodiment of the invention that the vectors of the invention be utilised with immunostimulatory agent. Preferably the immunostimulatory agent is administered at the same time as the nucleic acid vector of the invention and in preferred embodiments are formulated together. Such immunostimulatory agents include, (but this list is by no means exhaustive and does not preclude other agents): synthetic imidazoquinolines such as imiquimod [S-26308, R-837], (Harrison, et al. ‘Reduction of recurrent HSV disease using imiquimod alone or combined with a glycoprotein vaccine’, Vaccine 19:1820-1826, (2001)); and resiquimod [S-28463, R-848] (Vasilakos, et al. ‘Adjuvant activities of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as Interferon, particular Interferon alpha, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial immunostimulatory proteins such as Cholera Toxin, E. coli Toxin and mutant toxoids thereof can be utilised.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

Also provided are the use of a polynucleotide according to the invention, or of a vector according to the invention, in the treatment or prophylaxis of MUC-1 expressing tumour or metastases.

The present invention also provides methods of treating or preventing MUC-1 expressing tumour, any symptoms or diseases associated therewith including metastases, comprising administering an effective amount of a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversly the priming may be with the viral vector or with a protein formulation typically a protein formulated in adjuvant and the boost a DNA vaccine of the present invention.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

A preferred promoter element is the CMV immediate early promoter devoid of intron A, but including exon 1. Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full-length vaccinia constructs have been unsuccessful.

Bacterial vectors may also be employed, for example attenuted Salmonella, or Listeria may be used as a bacterial vector. The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polypeptide according to the invention include mammalian HEK293T, CHO, HeLa, 293 and COS cells. Preferably the cell line selected will be one which is not only stable, but also allows for mature glycosylation and cell surface expression of a polypeptide. Expression may be achieved in transformed oocytes. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

The invention further provides a method of vaccinating a mammalian subject which comprises administering thereto an effective amount of such a vaccine or vaccine composition. Most preferably, expression vectors for use in DNA vaccines, vaccine compositions and immunotherapeutics will be plasmid vectors.

DNA vaccines may be administered in the form of “naked DNA”, for example in a liquid formulation administered using a syringe or high pressure jet, or DNA formulated with liposomes or an irritant transfection enhancer, or by particle mediated DNA delivery (PMDD). All of these delivery systems are well known in the art. The vector may be introduced to a mammal for example by means of a viral vector delivery system.

The compositions of the present invention can be delivered by a number of routes such as intramuscularly, subcutaneously, intraperitonally or intravenously, muscosal such as the intranasal route.

In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

In an alternative embodiment, nucleotides of the present invention maybe administered by micro needles, which may have the DNA coated onto the needle or deliver the composition from a reservoir. The vectors which comprise the nucleotide sequences encoding antigenic peptides are administered in such amount as will be prophylactically or therapeutically effective. The quantity to be administered is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the weight of the patient being immunised and the route of administration.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. It is further possible that administration is required regularly for a longer period of time, whilst the progression of the disease is monitored. For example, for chronic cancer or other chronic conditions, monthly administration over a longer period than 18 months may be required. It is conceivable that regular administration for the lifetime of the patient may be needed for some patients/disease conditions. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

### EXAMPLES

1. Introduction MUC1 CODON Modification

Approach

Although MUC1 is a human gene with a RSCU (otherwise known as codon coefficient index (CI)) of 0.535, codon modification will further improve codon index and expression. This is particularly important in the clinical setting where dose may be limiting. A second advantage is that manipulation of the codon usage will reduce the potential for recombination between a MUC1 immunotherapeutic and the MUC1 locus in the genome. This is important in the clinical setting where recombination may lead to the integration of the plasmid into the genome.

1.1 Sequence Design

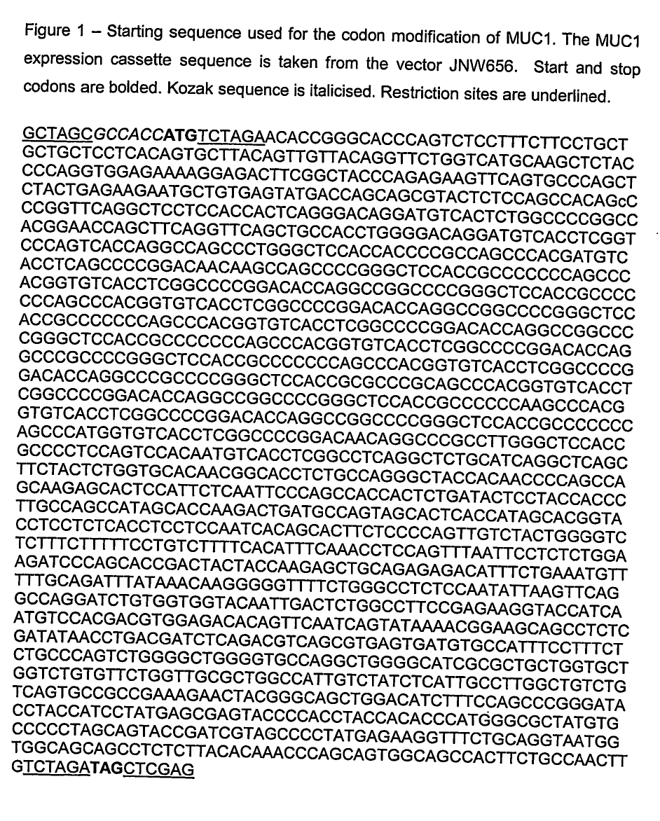

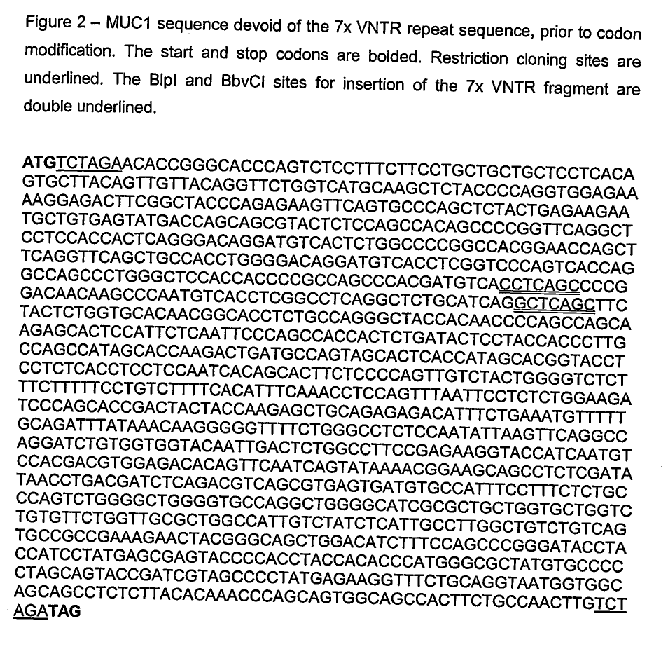

The starting sequence for the modification of MUC1 is shown in FIG. 1. This is derived from the plasmid JNW656 and represents the entire coding sequence of a MUC1 expression cassette containing seven VNTR repeat units. Prior to codon modification and because of previous difficulties in building up VNTR repeat units from oligonucleotides, a virtual MUC1 sequence devoid of VNTR repeats was created (FIG. 2). This sequence has a CI value of 0.499. The strategy was to codon optimise the non-VNTR sequences of MUC1 and then using restriction enzyme sites engineered into the codon modified sequence, re-insert the 7× VNTR fragment.

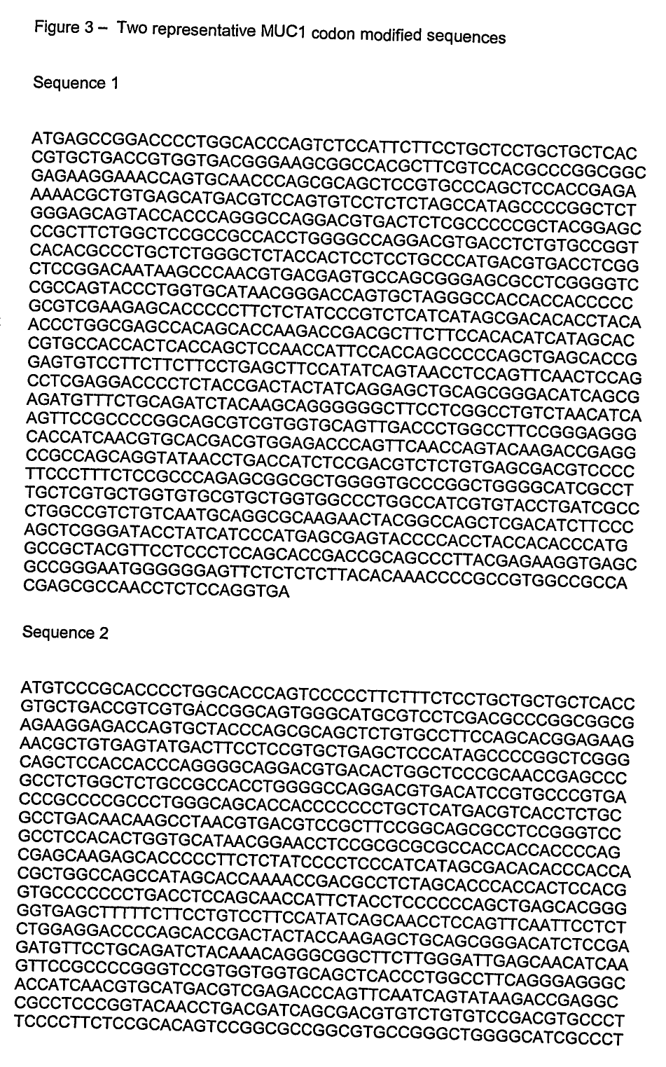

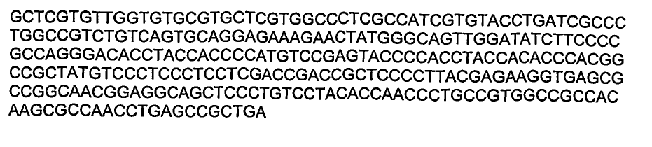

Using the Syngene programme, a selection of virtual codon modified sequences was obtained (FIG. 3) based upon the virtual MUC1 sequence in FIG. 2. Table 1 shows a comparison of the CI values for the starting MUC1 sequence and two representative codon modified sequences.

TABLE 1
Codon coefficient indices for MUC1 modified sequences

| Sequence | Codon coefficient index (CI) |
|---|---|
| MUC1 (devoid of 7x VNTR fragment) | 0.499 |
| Codon modified sequence 1 | 0.711 |
| Codon modified sequence 2 | 0.745 |

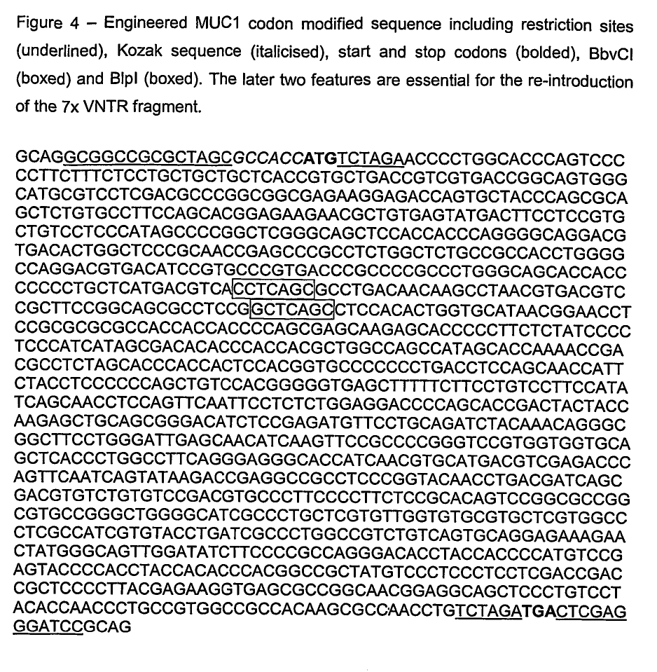

In addition to the codon medication, all sequences were also screened for restriction enzyme cloning sites. On the basis of the highest CI value and a favourable restriction enzyme site profile, sequence 2 was selected. To facilitate cloning and expression, the following changes were made to the sequence (see FIG. 4)

1) 5′ and 3′ cloning sites were added (NheI, XbaI, XhoI, NotI and BamHI)

2) A Kozak sequence (GCCACC) was inserted 5′ of the initiating ATG start codon.

3) Two inappropriate BlpI sites were removed by silent mutations at codons 64 (AGC→TCC) and 209 (AGC→TCC).

4) A rare Leucine codon was removed by the following mutation at codon 259 (TTG→CTG)

5) A Bpu10I/BbvCI site was re-introduced (see FIG. 4, boxed region) to facilitate cloning of 7× VNTR fragment

6) A BlpI site was re-introduced (see FIG. 4, boxed region) to facilitate cloning of 7× VNTR region

This engineered sequence is shown in FIG. 4 and has a CI value of 0.735. The Syngene programme was used to fragment this sequence into 52-60-mer oligonucleotides with a minimum overlap of 20 bases.

1.2 Oligo Build

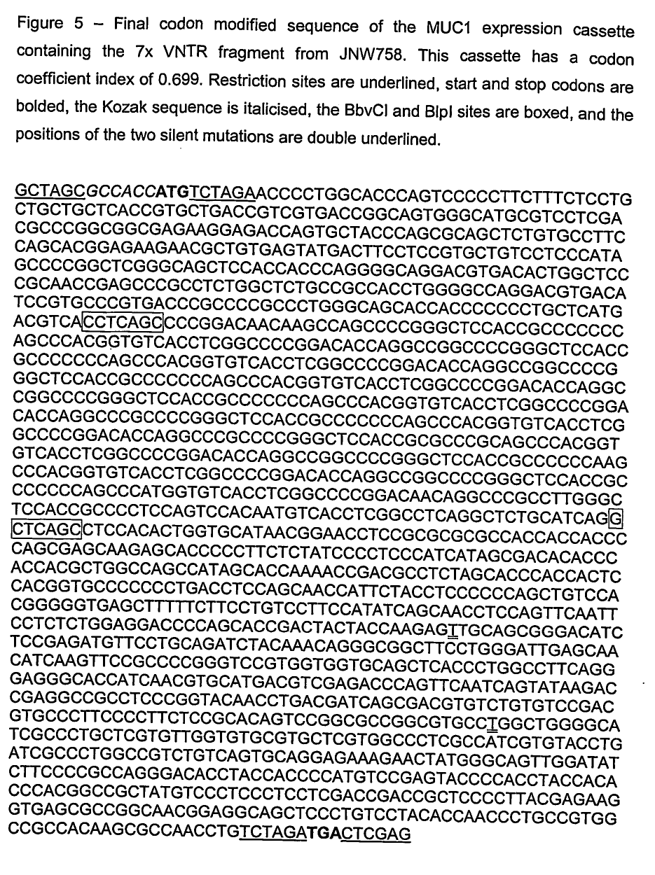

Using a two-step PCR protocol, the overlapping primers were first assembled using the conditions below. This generates a diverse population of fragments. The full-length fragment was recovered/amplified using the 5′ and 3′ terminal primers. The resulting PCR fragment was excised from an agarose gel, purified, restricted with NheI and XhoI and cloned into pVAC. Positive clones were identified by restriction enzyme analysis and sequence verified. The validated vector was labelled JNW749. The codon modified sequence of MUC1 in JNW749 contains two silent mutations (highlighted in FIG. 5) due to the error-prone nature of the oligonucletoide build-up.

Assembly Reaction—PCR Conditions

Reaction Mix:

1× Pfx buffer

1 μl Oligo pool

0.5 mM dNTPs

Pfx polymerase (5U)

1 mM MgSO₄

Total volume=50 μl

1. 94° C. 30 s
2. 40° C. 120 s
3. 72° C. 10 s
4. 94° C. 15 s
5. 40° C. 30 s
6. 72° C. 20 s + 3 s/cycle
7. Cycle to step 4, 25 times
8. Hold at 4° C.

Recovery Reaction—PCR Conditions
Reaction Mix:
1× Pfx buffer
10 μl assembly reaction mix
0.625 mM dNTPs
50 pmol 5′ terminal primer
50 pmol 3′ terminal primer
Pfx polymerase (5U)
1 mM MgSO₄
1× Pfx Enhancer

Total volume=50 μl

1. 94° C. 45 s
2. 60° C. 30 s
3. 72° C. 120 s
4. Cycle to step 1, 25 times
5. 72° C. 240 s
6. Hold at 4° C.

1.3 Re-Introduction of 7× VNTR Fragment

JNW749 contains a codon-modified MUC1 expression cassette devoid of the 7× VNTR unit. The 7× VNTR cassette was excised from JNW656 on a BlpI/BbvCI cassette and ligated into JNW749 previously restricted with BlpI and BbvCI. Following restriction enzyme analysis and sequence verification, a clone labelled JNW758 was selected for further analysis. The sequence of the MUC1 cassette in JNW758 is shown in FIG. 5. The final CI value of the MUC1 expression cassette in JNW758 is 0.699 which represents a substantial increase over the starting value of 0.535

1.4 Comparison of Expression of MUC1

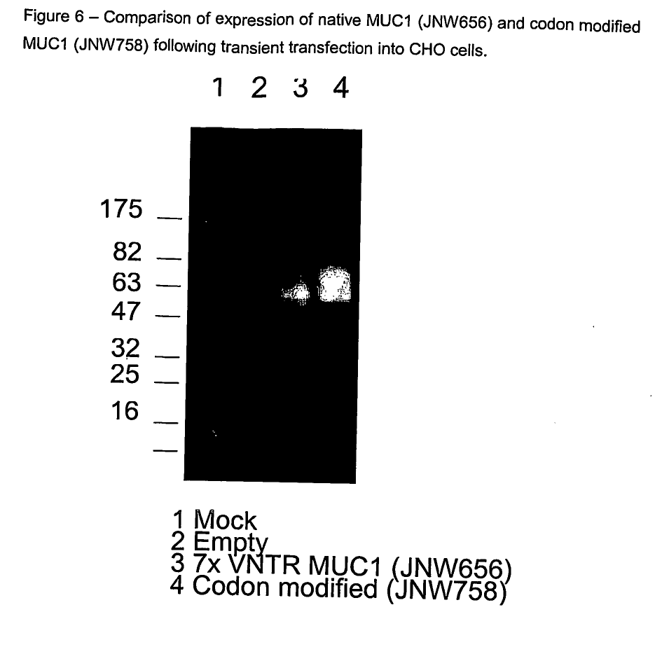

The expression of MUC1 from the vectors JNW656 (native MUC1) and JNW758 (codon modified MUC1) were compared following transient transfection into CHO cells. Using flow cytometric analysis (FACS), the percentage of cells expressing MUC1 at their surface is very similar between the native (13.2% for JNW656) and codon modified cassettes (18.1% for JNW758). When analysed by Western blot (FIG. 6), the results suggest that the expression of codon modified MUC1 is moderately enhanced when compared to the native MUC1. MUC1 expression on the Western blot was quantified by densitometry analysis using the Area Density Tool (Labworks, UVP Ltd, UK). MUC1 expression from JNW656 (native MUC1) gave an arbitrary spot density value of 48527, whilst the codon modified MUC1 (JNW758) gave a value of 94839, suggesting that the expression of codon modified MUC1 is enhanced approximately 2-fold when compared to the native 7× VNTR MUC1

1.5 DNA Similarity

Pair distances following alignment ClustalV (Weighted) of the starting sequence of MUC1 (from JNW656) and the codon modified sequence (from JNW758) confirms that the codon modified sequence is 82.8% similar to the original MUC1 sequence. Similarity of the same sequences devoid of the 7× VNTR region (between the BbvCI and BlpI sites) following ClustalV alignment is further reduced to 75.1%.

1.6 Comparison of Cellular Responses to 7× VNTR MUC1 and Codon Modified 7× VNTR MUC1

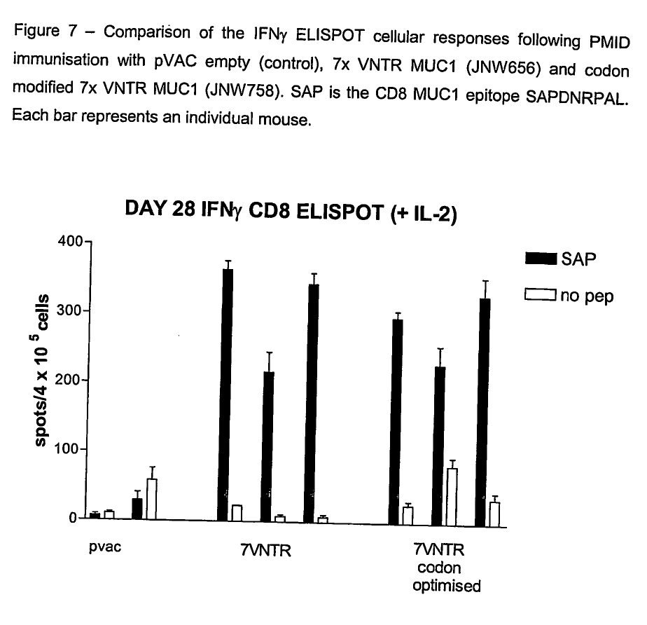

The cellular response following immunisation with pVAC (empty vector), JNW656 (7× VNTR MUC1) and JNW758 (codon modified 7× VNTR MUC1) were assessed by ELISPOT following a primary immunisation at day 0 and a boost at day 21. Assays were carried out 7 days post boost using the CD8 peptide SAPDNRPAL (SAP). FIG. 7 shows that the IFNγ production following re-stimulation of splenocytes with the SAP peptide and IL-2 is equivalent in groups immunised with either 7× VNTR MUC1 or codon modified 7× VNTR MUC1.

In conjunction with the results from the Western blot, these data suggest that codon modified 7× VNTR MUC1 compares favorably to native 7× VNTR MUC1 expression and immunogenicity and has significant advantages in terms of the reduced potential for recombination with the genomic MUC1 sequence.

1.7 Additional Methods

Methods for carrying out transient transfection assays MUC1 expression from various DNA constructs may be analysed by transient transfection of the plasmids into CHO (Chinese hamster ovary) cells followed by either Western blotting on total cell protein, or by flow cytometric analysis of cell membrane expressed MUC1. Transient transfections may be performed with the Transfectam reagent (Promega) according to the manufacturer's guidelines. In brief, 24-well tissue culture plates may be seeded with 5×10⁴ CHO cells per well in 1 ml DMEM complete medium (DMEM, 10% FCS, 2 mM L-glutamine, penicillin 100 IU/ml, streptomycin 100 μg/ml) and incubated for 16 hours at 37° C. 0.5 μg DNA may be added to 25 μl of 0.3M NaCl (sufficient for one well) and 2 μl of Transfectam added to 25 μl of Milli-Q. The DNA and Transfectam solutions should be mixed gently and incubated at room temperature for 15 minutes. During this incubation step, the cells should be washed once in PBS and covered with 150 μl of serum free medium (DMEM, 2 mM L-glutamine). The DNA-Transfectam solution then should be added drop wise to the cells, the plate gently shaken and incubated at 37° C. for 4-6 hours. 500 μl of DMEM complete medium should then be added and the cells incubated for a further 48-72 hours at 37° C.

1.8 Flow Cytometric Analysis of CHO Cells Transiently Transfected with MUC1 Plasmids

Following transient transfection, the CHO cells were washed once with PBS and treated with a Versene (1:5000)/0.025% trypsin solution to transfer the cells into suspension. Following trypsinisation, the CHO cells were pelleted and resuspended in FACS buffer (PBS, 4% FCS, 0.01% sodium azide). The primary antibody, ATR1 was added to a final concentration of 15 μg/ml and the samples incubated on ice for 15 minutes. Control cells were incubated with FACS buffer in the absence of ATR1. The cells were washed three times in FACS buffer, resuspended in 100 μl FACS buffer containing 10 μl of the secondary antibody goat anti-mouse immunoglobulins FITC conjugated F(ab′)₂ (Dako, F0479) and incubated on ice for 15 minutes. Following secondary antibody staining, the cells were washed three times in FACS buffer. FACS analysis was performed using a FACScan (Becton Dickinson). 1000-10000 cells per sample were simultaneously measured for FSC (forward angle light scatter) and SSC (integrated light scatter) as well as green (FL1) fluorescence (expressed as logarithm of the integrated fluorescence light). Recordings were made excluding aggregates whose FCS were out of range. Data were expressed as histograms plotted as number of cells (Y-axis) versus fluorescence intensity (X-axis).

1.9 Western Blot Analysis of CHO Cells Transiently Transfected with MUC1 Plasmids

The transiently transfected CHO cells were washed with PBS and treated with a Versene (1:5000)/0.025% trypsin solution to transfer the cells into suspension. Following trypsinisation, the CHO cells were pelleted and resuspended in 50 μl of PBS. An equal volume of 2× TRIS-Glycine SDS sample buffer (Invitrogen) containing 50 mM DTT was added and the solution heated to 95° C. for 5 minutes. 1-20 μl of sample was loaded onto a 4-20% TRIS-Glycine Gel 1.5 mm (Invitrogen) and electrophoresed at constant voltage (125V) for 90 minutes in 1× TRIS-Glycine buffer (Invitrogen). A pre-stained broad range marker (New England Biolabs, #P7708S) was used to size the samples. Following electrophoresis, the samples were transferred to Immobilon-P PVDF membrane (Millipore), pre-wetted in methanol, using an Xcell III Blot Module (Invitrogen), 1× Transfer buffer (Invitrogen) containing 20% methanol and a constant voltage of 25V for 90 minutes. The membrane was blocked overnight at 4° C. in TBS-Tween (Tris-buffered saline, pH 7.4 containing 0.05% of Tween 20) containing 3% dried skimmed milk (Marvel). The primary antibody (ATR1) was diluted 1:100 and incubated with the membrane for 1 hour at room temperature. Following extensive washing in TBS-Tween, the secondary antibody was diluted 1:2000 in TBS-Tween containing 3% dried skimmed milk and incubated with the membrane for one hour at room temperature. Following extensive washing, the membrane was incubated with Supersignal West Pico Chemiluminescent substrate (Pierce) for 5 minutes. Excess liquid was removed and the membrane sealed between two sheets of cling film, and exposed to Hyperfilm ECL film (AmershamPharmaciaBiotech) for 1-30 minutes.

### Example 2

### Comparison of Cellular Responses to 7VNTR-MUC-1-PADRE-C and Codon Modified 7VNTR-MUC-1-PADRE-C

2.1 Construction of Codon-Optimised MUC-1 Padre

Construction of MUC1 Expression Cassettes Fused to the PADRE Helper Epitope

Three MUC1 designs containing the PADRE helper epitope (see Immunity (1994) 1(9):751-761) were constructed. PADRE is a pan-DR binding epitope containing a polyalanine backbone with bulky/charged residue substitutions at positions accessible to the T cell receptor. A C-terminal fusion was generated by first inserting a short linker into pVAC1. The linker was created by annealing the two primers PADREFOR and PADREREV and cloning the linker into pVAC1 via the NheI and XhoI sites, generating vector JNW800. Into JNW800, the 7× VNTR MUC1 expression cassette from JNW656 (7× VNTR MUC1) and JNW758 (codon optimised 7× VNTR MUC1,) was inserted by excising the MUC1 cassette on an XbaI fragment and cloning into the XbaI site, generating the following two vectors

7× VNTR MUC1 C-term PADRE: JNW810

7× VNTR MUC1 (codon optimised) C-term PADRE: JNW812

The sequencing of the MUC1 expression cassette and PADRE epitope from JNW810 and JNW812.

A third vector in which the PADRE sequence is inserted at the extreme C-terminus and also at a second position just after the signal sequence of MUC1, was constructed. The rationale for inserting the N-terminal PADRE epitope downstream of the signal sequence was to avoid the epitope being cleaved off as part of the natural post-translational processing of the MUC1 peptide (see Biochem. Biophys. Res. Comm (2001) 283: 715-720 for details of sites of cleavage in MUC1). The vector was constructed in a 2-stage process. Firstly, the N-terminal sequence of MUC1 containing both the N-terminal and C-terminal PADRE epitopes was generated in silico and then built by PCR using overlapping oligos (as described). The PCR fragment was inserted into pVAC1 via the NheI-XhoI sites and sequence validated, generating plasmid JNW802. The C-terminal portion of codon optimised 7× VNTR MUC1 was isolated from JNW758 on a BbcVI-XbaI fragment and cloned in to JNW802, thus re-creating the 7× VNTR MUC1 expression cassette containing two PADRE epitopes. This vector is labelled 7× VNTR MUC1 (codon optimised) C/N′PADRE or JMW814.

2.2 30 C57 Mice Were Evaluated in Five Groups (six mce/group)

| | | |
|---|---|---|
| A. | PVac 7 VNTR | JNW656 |
| B. | pVac 7 VNTR PADRE C (codon-optimised) | JNW812 |
| C. | pVac 7 VNTR PADRE C (wild type) | JNW810 |
| D. | pVav 7 VNTR PADRE C/N′ (codon-optimised) | JNW814 |

E. pVac Empty

Each animal was immunised by particle mediated immunisation with the expression plasmid at day 0, 12 and 42 (1 μg MUC-1 DNA+0.5 μg 1L-2) cellular immune responses were assessed at day 28 and day 49.

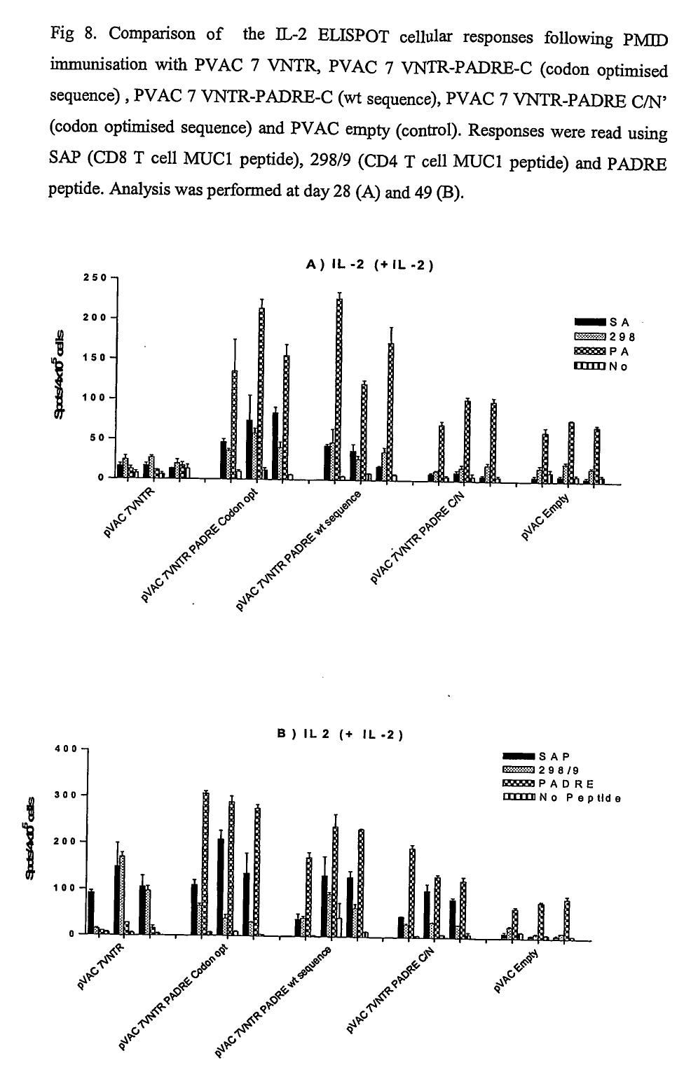

Results are shown in FIGS. 8 A and B.

### CONCLUSION

The cellular responses following immunisation with PVAC 7VNTR, PVAC 7VNTR-PADRE-C codon optimised sequence, PVAC 7VNTR-PADRE-C wt sequence, PVAC 7VNTR-PADRE C/N′ codon optimised sequence were assessed by ELISPOT following a primary immunisation at day 0 and two boosts at day 21 and 42. Assays were carried out 7 days post boost using the MUC1 CD8 peptide (SAP), the MUC1 CD4 peptide (298/9) and the PADRE peptide. Results show that both CD4 and CD8 T cell MUC1 specific responses are similar (or slightly better) in the codon optimised construct than in the wt mice at day 28 and day 49, and are designed to avoid homologous recombination.

In conclusion the inclusion of codon optimised sequences within the MUC1 antigen improves protein expression, generates similar or slightly better immune responses when used in vivo and they are expected to have a better safety profile to use in a human clinical vaccine

1. A nucleic acid molecule encoding a MUC-1 derivative which is capable of raising an immune response in vivo, said response being capable of recognising a MUC-1 expressing tumour, wherein the nucleic acid has a RSCU value for the non-repeat region of at least 0.6 and has a level of identity of less than 85% in comparison with the MUC-1 VNTR nucleotide sequence shown in FIG. 9, with respect to the corresponding non-repeat region of wild type MUC-1.

2. A nucleic acid module as claimed in claim 1 wherein the RSCU is at least 0.65.

3. A nucleic acid molecule as claimed in claim 1 wherein the identity is less than 80%.

4. A nucleic acid molecule encoding a MUC-1 derivative as claimed in claim 1 having less than 15 perfect repeat units.

5. A nucleic acid molecule as claimed in claim 4 having no perfect repeats.

6. A nucleic acid molecule as claimed in claim 1 of which is devoid of a signal sequence.

7. A nucleic acid molecule as claimed in claim 1 that encodes one or more of the sequence from the group: FLSFHISNL; NSSLEDPSTDYYQELQRDISE; and NLTISDVSV.

8. A nucleic acid molecule as claimed in claim 1 additionally comprising a heterologous sequence that encodes a T-Helper epitope.

9. A nucleic acid molecule as claimed in claim 1 that is a DNA molecule.

10. A plasmid comprising the DNA molecule of claim 1.

11. A pharmaceutical composition comprising a nucleic acid as claimed in claim 1 and a pharmaceutical acceptable excipient, diluent or carrier.

12. A pharmaceutical composition as claimed in claim 11 wherein the carrier is a microparticle.

13. A pharmaceutical composition as claimed in claim 12 wherein the microparticle is gold.

14. A pharmaceutical composition as claimed in claim 11 additionally comprising an adjuvant.

15. A medicament comprising a nucleic acid as claimed in claim 1.

16. A method of treating or preventing MUC-1 expressing tumours comprising administering a medicament comprising a nucleic acid as claimed in claim 1.

17. A method of treating or preventing tumours, comprising administering a safe and effective amount of a nucleic acid as claimed in claim 1.

18. A medicament comprising a plasmid as claimed in claim 10.

19. A medicament comprising a pharmaceutical composition as claimed in claim 11.

20. A method of treating or preventing tumours, comprising administering a safe and effective amount of a plasmid as claimed in claim 10.
